(19) United States
(12) Patent Application Publication
(10) Pub. No.: US 2022/0005199 A1
(43) Pub. Date: Jan. 6, 2022

(54) IMAGE SEGMENTATION WITH KINEMATIC DATA IN A ROBOTIC SURGICAL SYSTEM

(71) Applicant: Asensus Surgical US, Inc., Durham, NC (US)

(72) Inventor: Kevin Andrew Hufford, Cary, NC (US)

(21) Appl. No.: 17/368,757

(22) Filed: Jul. 6, 2021

Related U.S. Application Data
(60) Provisional application No. 63/048,179, filed on Jul. 5, 2020.

(51) Int. Cl. G06T 7/11; G06T 7/168; A61B 34/00; A61B 34/20

(52) U.S. Cl. CPC G06T 7/11; G06T 7/168; A61B 2034/2065; A61B 34/20; A61B 34/70

(57) ABSTRACT

A system makes use of kinematic data from a robotic manipulator to aid in segmenting images of a robotically maneuvered surgical instrument at an operative site. A camera is positioned to capture image data of the surgical instrument at the surgical site. At least one processor receives the image data as well as kinematic data from the robotic manipulator. The kinematic data is used to define regions of interest in the images, and image segmentation is then carried out at the regions of interest to identify the surgical instruments in the image data.

### BACKGROUND

There are various types of surgical robotic systems on the market or under development. Some surgical robotic systems use a plurality of robotic arms. Each arm carries a surgical instrument, or the camera used to capture images from within the body for display on a monitor. Other surgical robotic systems use a single arm that carries a plurality of instruments and a camera that extend into the body via a single incision. Each of these types of robotic systems uses motors to position and/or orient the camera and instruments and to, where applicable, actuate the instruments. Typical configurations allow two or three instruments and the camera to be supported and manipulated by the system. Input to the system is generated based on input from a surgeon positioned at a master console, typically using input devices such as input handles and a foot pedal. Motion and actuation of the surgical instruments and the camera is controlled based on the user input. The image captured by the camera is shown on a display at the surgeon console. The console may be located patient-side, within the sterile field, or outside of the sterile field.

Advancing technologies make use of information acquired from the computer vision system as an input that can result in intelligent actions of a robotic surgical system. Optimizing such functions is enhanced by increased assurance of the fidelity of that data. In a robotic surgical system, there is a unique advantage provided by integrating information acquired not only from the endoscope, but also from the motion commands of the robotic arms as well.

This invention aims to make image segmentation for computer vision techniques more robust and responsive by making use of data from the motion commands of the robotic arms.

### BRIEF DESCRIPTION OF THE DRAWINGS

FIG. 1 depicts a field of view of surgical instruments at a surgical site as captured in an image by a camera, and shows detection of the boundaries of the surgical instruments and an increase in the boundary margin of detection;

FIG. 2 is similar to FIG. 1, and depicts transformation of the boundary is applied from accrued incremental motion of the manipulator to the next captured image, creating a region of interest;

FIG. 3 depicts the region of interest as transmitted to the computer vision algorithm;

FIGS. 4-5 depict use of the computer vision algorithm on the region of interest to finally detect the surgical instruments.

FIGS. 6 and 7 depict optional steps of cropping and rotating the images prior to application of the detection algorithm.

FIG. 8 is a schematic block diagram of an embodiment of the disclosed system.

### DETAILED DESCRIPTION

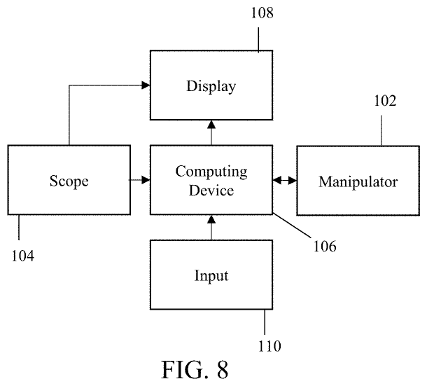

Referring to FIG. 8, in general, the system operates in conjunction with a robotic surgical system comprising at least one manipulator 102 holding a surgical instrument, and a camera (e.g. an endoscope) 104 whose video output is processed by at least one processing unit 106. At least one processor is configured for receiving the image output as well as kinematic data from the robotic manipulators 102. It includes a memory storing instructions for executing the various features described here, and a database associated with the processor. An image display 108 may be provided for displaying the images. User input devices 110 may also be included, such as, without limitation, vocal input devices, manual input devices (e.g. buttons, touch inputs, knobs, dials, foot pedals, eye trackers, trackers etc.), including input devices that are part of the surgeon console used by the surgeon to give input to the surgical system to command movement and actuation of surgical instruments carried by robotic manipulators.

The system uses kinematic data from motions of a robotic surgical system to aid in image segmentation for computer vision recognition of instruments at the surgical site. In the described methods, the one or more processors associated with the computer vision system receive image data captured by the camera. Kinematic data from robotic manipulators is used to provide input to a computer vision system to define or create regions of interest for image segmentation. Image segmentation is then performed in those regions of interest to identify surgical tools within those regions. These methods reduce latency and increase frame rate for surgical tool recognition, and they result in more robust computer vision system outputs, because solutions that do not coincide with instrument motion are rejected by definition (and may not even be seen by the computer vision system).

The systems/methods can perform computer vision of the (full) endoscope image to detect the surgical tool(s)/instrument(s) or its boundaries. This computer vision processing may utilize neural networks and/or other computer vision techniques such as, but not limited to: edge detection, shape recognition, region growing, active contour models (snakes), Haar cascades, scale-invariant feature transform (SIFT), speeded up robust features (SURF), or any combination thereof. In some implementations, fast algorithms for detecting linear-type objects may be used initially to define regions of interest, which are then passed to other algorithms (neural networks or otherwise) for robust classification to determine if they are in fact surgical tools.

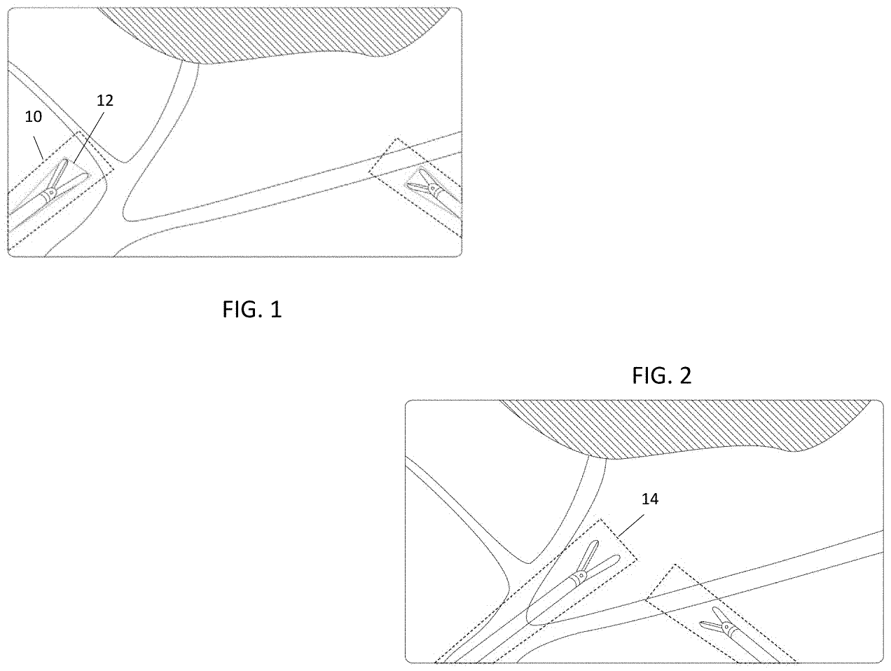

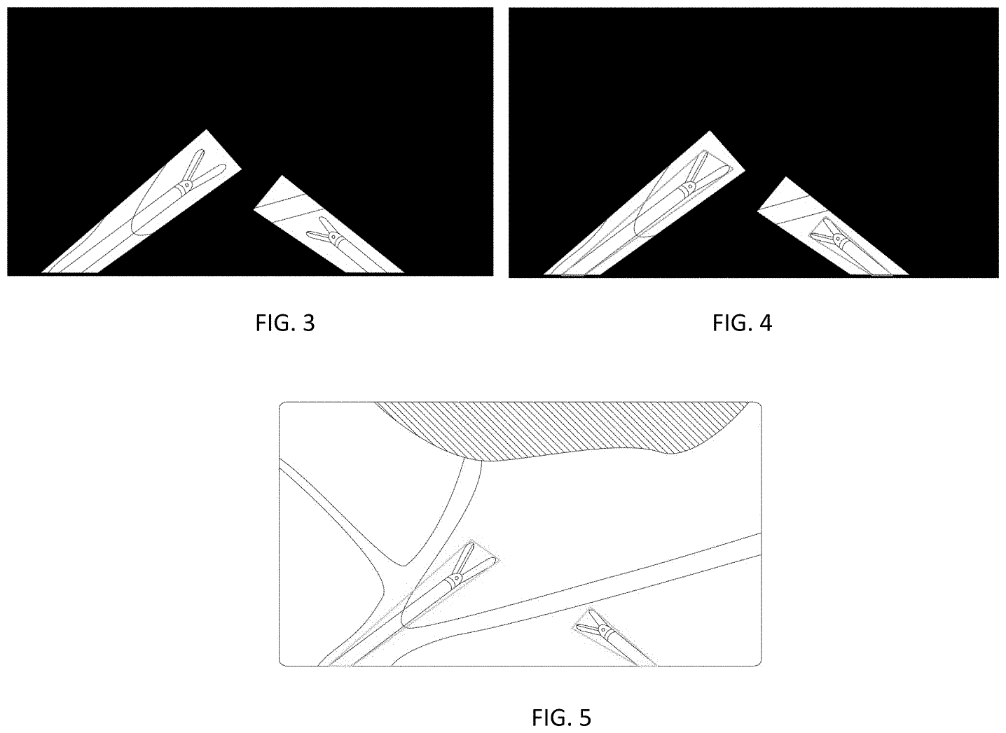

Referring to FIG. 1, the boundaries of the detected surgical instrument(s) are stored. These are identified by boundary 12 in FIG. 1. A transformation is applied to grow the boundary of the detected tool and increase the margin for detection in the next frame, which is identified by boundary 10 in FIG. 1. A transformation of this boundary is applied from the accrued incremental motion of the robotic manipulator from the current endoscopic image to a subsequent (or the next) endoscope image, creating a region of interest 14 (FIG. 2) which is then transmitted to the computer vision algorithm for final detection of the surgical tool (FIGS. 3-5).

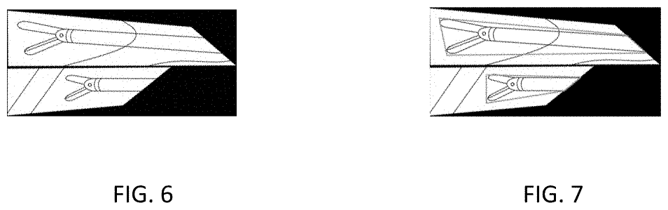

Referring to FIGS. 6 and 7, for significantly improved processing efficiency, in some implementations, the image is cropped and rotated, transmitting only the regions of interest to the detection algorithm, potentially using processor parallelization to improve performance even more. Once detection has occurred, the inverse of the transformation of the region of interest may be applied to determine the locations of the tools relative to the full surgical site image.

Once instrument detection has occurred, this information may be used in a variety of ways.

Interactions of this system with a 3D model of the surgical field may include, but are not limited to: updating the actual or predicted tool positions based on robotic manipulator motion, adjusting the transformations of the region of interest for each “eye” of a 3D stereo image, etc.

In various applications, it may be advantageous to use different modes of data as the “ground truth.” For example, computer vision might be applied for initial scene awareness, kinematic data used for responsive data, with computer vision then used as a double-check, and as a less-frequent update of soft-tissue structure locations.

The described technique may be used in a live application to improve the responsiveness of the system and/or may be used during training of neural networks, machine learning, artificial intelligence to reduce the machine learning training time.

Where manual laparoscopic instruments or other items besides robotically manipulated instruments may be introduced into the surgical field, image processing on only a restricted region of interest may not be the only suitable approach. In such cases a whole-field analysis may still have to be performed, but in implementations with more-limited computing resources, this may be done at a lower frame rate and/or resolution in parallel with the full-frame-rate analysis of the regions of interest.

Advantages provided by the disclosed system and method include:
Reduced latency for computer vision algorithm by only processing a region-of-interest rather than the entire scene at full frame rate
Better system response to events/motion in the surgical image (avoidance, no-fly zones, semi-autonomous motion, training of machine learning, etc.)

I claim:

1. A system for segmenting images of surgical instruments from image data, comprising:
at least one manipulator holding a surgical instrument, the surgical instrument positionable at a surgical site in a body cavity;
a camera positionable to capture image data of the surgical instrument at the surgical site;
at least one processor is configured for receiving the image data and kinematic data from the robotic manipulators, the processor including a memory storing instructions that, when executed, use the kinematic data to define regions of interest for image segmentation, and then perform image segmentation at the regions of interest to identify the surgical instruments in the image data.
